(19) United States
(12) Patent Application Publication
(10) Pub. No.: US 2024/0326047 A1
(43) Pub. Date: Oct. 3, 2024

(54) SINGLE MOLECULE/SINGLE CELL DETECTION CHIP

(71) Applicant: SHENZHEN CAS-ENVISION MEDICAL TECHNOLOGY CO.,LTD, Shenzhen (CN)

(72) Inventors: TIANZHUN WU, Shenzhen (CN); SAISAI ZHAO, Shenzhen (CN); HAOFAN REN, Shenzhen (CN)

(21) Appl. No.: 18/739,740

(22) Filed: Jun. 11, 2024

Related U.S. Application Data
(63) Continuation of application No. PCT/CN2022/126240, filed on Oct. 19, 2022.

(30) Foreign Application Priority Data
Oct. 20, 2021 (CN) 202111224380.3

(51) Int. Cl. B01L 3/00

(52) U.S. Cl. CPC B01L 3/502753; B01L 2200/0689; B01L 2300/0681; B01L 2300/0819; B01L 2300/1811; B01L 2400/022

(57) ABSTRACT

A single molecule/single cell detection chip, including: a micropore array, comprising multiple micropore arrays for dividing the test solution into test target droplets; a detection IC circuit, located below the micropore array, including: a detection unit: comprising multiple detection subunits set one-to-one correspondence with multiple micropores, multiple detection subunits connected to a main control unit for measuring the fluorescence intensity of target nucleic acid/protein molecule/cell, and sending the raw measurement results to the main control unit; Main control unit: used for power management, controls the detection unit through row and column selection, receiving raw results, and generating final detection results based on the raw measurement results. This present application integrates the functions of target droplet generation, arraying, nucleic acid/protein molecule/cell detection, photoelectric detection, and data processing through the detection chip. It simplifies the overall structure of the chip, improves reaction speed and detection performance, and enhances chip stability.

### CROSS-REFERENCE TO RELATED APPLICATIONS

This application is a continuation of co-pending International Patent Application No. PCT/CN2022/126240, filed on Oct. 19, 2022, which claims the priority and benefit of Chinese patent application number 202111224380.3, filed on Oct. 20, 2021 with China National Intellectual Property Administration, the entire contents of which are incorporated herein by reference.

### TECHNICAL FIELD

This application pertains to the field of single molecule and single cell detection, particularly relating to a nucleic acid molecule/protein molecule/cell detection chip.

### BACKGROUND

Currently, the biomarkers that can be detected are just the tip of the iceberg, and a large number of low-abundance molecules are still awaiting discovery by highly sensitive detection instruments and methods. To detect extremely trace amounts of early biomarkers with high sensitivity, the emergence of Single Molecule Detection (SMD) since 2000 has been expected to bring about a revolutionary technological breakthrough. It completely changes the approach by randomly dispersing trace biomarkers across arrays of tens or hundreds of thousands, transforming traditional solution-based and ensemble-averaged molecular signals and comparative analysis with reference templates into high-throughput discrete signals (0 or 1, relative to a set detection threshold) for absolute counting and quantification. The higher the throughput, the higher the sensitivity. It is not only the ultimate goal long pursued in the field of analytical detection, an ultimate means of absolute quantification, but also an important means for early disease diagnosis and screening, greatly advancing molecular diagnostics towards digitization and informatization, fully reflecting the deep integration trend of IT (Information Technology)+BT (Biotechnology). The current mainstream and extended technologies include digital PCR, digital ELISA (single-molecule immunoassay), and single-cell analysis, all of which can be classified as digital single molecule/single cell detection, creating a new generation of nucleic acid, immune, and cell analysis technologies. These three technologies have moved from the laboratory to industrialization, with products from several foreign giants already on the market and widely used in scientific research and clinical testing, including Bio-Rad, Fluidigm, Life Technologies, and RainDance. There are also many research institutions and in vitro diagnostic-related companies actively developing digital PCR, digital ELISA, and single-molecule cell technology, promoting industry development. Currently, these three types of analytical instruments usually require microfluidic chips with different designs, combined with precise and complex fluid control, optical detection, algorithms, software, etc., making them expensive to produce, costly to purchase, and poor in stability.

If we could integrate core functions such as droplet generation, biochemical reactions, fluorescence signal detection, and digital counting into a biochip, it would greatly simplify the functions of complex optomechanical systems and reduce costs. The integrated biochip can be mass-produced and reliably prepared using mature semiconductor processes, disposable after use, and the cost can be sufficiently low as long as the quantity is enough.

Currently, there is no patented technology that can support the integration of dPCR, dELISA, and single-cell analysis chip functions based on the same chip architecture.

### SUMMARY

To achieve the above objectives, the following technical solutions have been adopted in this application:

The application provides a single molecule/single cell detection chip, which includes:

Micropore array, set on the surface of the single molecule/single cell detection chip, comprising multiple micropores. These micropores are used to divide the test solution into multiple test target droplets, which include a reaction solution and at most one target nucleic acid molecule/protein molecule/cell. The target nucleic acid molecule/protein molecule/cell emits light when combined with the reaction solution.

Detection IC circuit, located below the micropore array, it includes:

Detection unit, comprising multiple detection sub-units that are set one-to-one corresponding to the multiple micropores and connected to the main control unit. The detection sub-units are used to identify test droplets in which the target nucleic acid molecule/protein molecule/cell emits light with an intensity greater than a first threshold, obtain raw measurement results, and send these raw measurement results to the main control unit.

Main control unit, used for power management, clock management, controlling the detection sub-units, receiving the raw measurement results, generating final detection results based on all the raw measurement results, and outputting the final detection results to the external circuit of the chip.

It can be seen that this application integrates functions such as amplification, detection, and data processing through the detection chip, simplifying the structure of the single molecule/single cell detection chip and enhancing its stability.

In some embodiments, the multiple micropore arrays are orderly arranged on the micropore array, and all the pore walls of the micropores are perpendicular to the bottom of the micropores; or, all the pore walls of the micropores form an acute or obtuse angle with the bottom of the micropores.

In some embodiments, the micropore array includes multiple droplet areas, with the multiple micropores distributed over these droplet areas; the test solution flows along a predetermined direction and covers the multiple droplet areas, forming a test droplet array.

In some embodiments, the inner side surface of the micropores is hydrophilic, and the bottom of the micropores is hydrophilic or hydrophobic

In some embodiments, the micropore array is made of inert materials and obtains hydrophilicity or hydrophobicity through physical modification or chemical modification.

In some embodiments, the detection sub-unit includes a stacked arrangement of a filter layer, heating electrode, detection circuit, and auxiliary circuit;

The filter layer is set below the corresponding micropore, composed of a first refractive layer and a second refractive layer stacked together, used to filter the incident excitation light of the micropore. After droplet amplification, the longer-wavelength light emission passes through the filter layer to reach the detection unit. The refractive index of the first refractive layer is different from that of the second refractive layer.

The heating electrode is set between the filter layer and the detection circuit, or between the micropore and the filter layer, used to heat the test droplet to the target temperature for isothermal amplification, to carry out the reaction of nucleic acid molecules/protein molecules/cells; or to perform multiple temperature cycles for the variable temperature amplification reaction of nucleic acids.

The detection circuit includes one or more photodetectors, used to receive row and column gating instructions and control commands. When these photodetectors receive a light signal, they generate and send the raw measurement results to the main control unit.

The photodetectors can be photodiodes or avalanche diodes, or other sensors capable of photoelectric conversion.

The auxiliary circuit includes a temperature sensing circuit. The thermosensitive element of the temperature sensing circuit is set close to the micropore or inside the main control unit. It is used to read the temperature signals of one or more detection circuits and output them through the main control circuit to the external circuit.

In some embodiments, the auxiliary circuit also includes multiple metal connection lines. These metal connection lines are respectively set between the heating electrode, temperature sensor, and the detection circuit, making the heating electrode, temperature sensor, and the detection circuit electrically connected to the main control unit.

In some embodiments, the filter layer or heating electrode is equipped with microlenses for converging the fluorescence emitted from the micropores.

In some embodiments, the main control unit includes a power management circuit, clock management circuit, row and column selection circuit, signal readout circuit, signal processing circuit, and I/O interface circuit.

The power management circuit is responsible for converting the external power supply of the chip into one or more DC levels within the chip.

The clock management circuit is designed to receive and process the clock signal provided externally to the chip, serving as the time base for the chip's internal digital circuitry.

The row and column selection circuit, connected to the power management circuit, sends gating instructions to select the appropriate row and column positions of the detection sub-units.

The signal readout circuit, linked to the power management circuit, reads all light signals passing through the filter layer and converts them into electrical signals via the photodetectors.

Alternatively, the signal readout circuit includes a preprocessing circuit, connected to the main control unit, which averages and denoises the digital electrical signals multiple times, or compresses the signals.

The I/O interface circuit, connected to both the signal readout and temperature sensing circuits, inputs external power, clock, and control signals into the chip. It also transmits the digital signals from the signal readout circuit and the temperature signals from the temperature sensing circuit to the external circuit of the chip in digital form.

In some embodiments, the micropores are created using a polymer through-hole array processed by laser machining or a fiber bundle slice etched by wet etching.

In certain implementations, the filter layer or heating electrode is equipped with microlenses designed to focus the light emitted from the micropores.

In some implementations, the main control unit includes a power management circuit, clock management circuit, row and column selection circuit, signal readout circuit, signal processing circuit, and I/O interface circuit;

The power management circuit is responsible for converting the external power supply to one or more DC levels inside the chip.

The clock management circuit receives and processes the external clock signal to serve as the time base for the internal digital circuitry of the chip.

The row and column selection circuit, connected to the power management circuit, sends gating instructions to select the appropriate row and column positions of the detection sub-units.

The signal readout circuit, linked to the power management circuit, reads the raw measurement results outputted by the detection circuit and converts them into digital electrical signals.

The signal readout circuit also includes a preprocessing circuit, connected to the main control unit, which averages and denoises the digital electrical signals multiple times, or compresses the signals.

The I/O interface circuit, connected to both the signal readout and temperature sensing circuits, inputs external power, clock, and control signals into the chip. It also transmits the digital signals from the signal readout circuit and the temperature signals from the temperature sensing circuit to the external circuit of the chip in digital form.

In some implementations, the micropores are fabricated using MEMS technology compatible with CMOS processes, or using precisely machined regular micro-through-hole arrays, and are bonded and aligned to the main control unit, allowing the signal readout circuit to read the optical signals from the micropore array one by one.

In some implementations, the detected light can be visible light, fluorescent luminophores, upconversion luminescence, rare earth element luminescence, or quantum dot luminescence detection.

Compared to existing technologies, the single molecule/single cell detection chip provided by this application has the following beneficial effects:

This application integrates functions such as amplification, identification, and data processing through the detection chip, simplifying the structure of the biomimetic detection chip and enhancing its stability. It utilizes mature CIS and MEMS processes, allowing for mass production at low cost and high quality control. The chip integrates liquid sampling, droplet generation, photoelectric detection, and temperature control modules on a silicon base. It is easily expandable, with the capability to increase the number of droplet areas to achieve high-throughput human samples and light detection channels.

### BRIEF DESCRIPTION OF DRAWINGS

FIG. 1 shows the structural framework of the detection IC circuit provided by this application.

FIG. 2 illustrates the arrangement of the micropores as provided by this application.

FIG. 3 is an exploded view of the structure of Example 1 of the single molecule/single cell detection chip provided by this application.

FIG. 4 is an exploded view of the structure of Example 2 of the single molecule/single cell detection chip provided by this application.

FIG. 5 shows a structural diagram of one embodiment of the micropore provided by this application.

FIG. 6 shows a structural diagram of another embodiment of the micropore provided by this application.

FIG. 7 is a structural diagram of the single molecule/single cell detection chip provided by this application.

FIG. 8 illustrates a bright field arrangement of one type of micropore in the single molecule/single cell detection chip provided by this application.

FIG. 9 is a diagram of nucleic acid molecule detection by digital PCR in the single molecule/single cell detection chip provided by this application.

FIG. 10 is a diagram of protein molecule detection by digital ELISA in the single molecule/single cell detection chip provided by this application.

FIG. 11 is a diagram of single cell detection in the single molecule/single cell detection chip provided by this application.

### DESCRIPTION OF EMBODIMENTS

To facilitate a better understanding of this application's proposal by those skilled in the art, the following will provide a clear and complete description of the technical solutions in the embodiments of this application, in conjunction with the accompanying drawings. It is evident that the described embodiments are only a part of the embodiments of this application and not all of them. Based on the embodiments in this application, all other embodiments obtained by those of ordinary skill in the art without creative efforts fall within the scope of protection of this application.

The terms “first,” “second,” and the like in the specification and claims of this application and the above-mentioned drawings are used to distinguish between different objects, not to describe a specific order. Moreover, the terms “comprising” and “having,” and any of their variations, are intended to cover a non-exclusive inclusion. For example, a process, method, system, product, or device that includes a series of steps or units is not limited to the listed steps or units but optionally includes steps or units not listed, or optionally includes other steps or units inherent to these processes, methods, products, or devices.

The mention of “embodiments” in this document means that a particular feature, structure, or characteristic described in connection with the embodiment is included in at least one embodiment of the application. The phrase does not necessarily refer to the same embodiment throughout the specification, nor is it an independent or alternative embodiment exclusive of other embodiments. Those skilled in the art will explicitly and implicitly understand that the embodiments described herein can be combined with other embodiments.

In the context of this application, “at least one” refers to one or more, with multiple indicating two or more. The term “and/or” describes a relationship between associated objects, indicating three possible relationships. For example, A and/or B can mean: A exists alone, both A and B exist together, or B exists alone, where A and B can be singular or plural. The character “/” typically signifies an “or” relationship between the associated objects. “At least one (item)” or similar expressions refer to any combination of these items, including any combination of single or multiple items. For instance, at least one of a, b, or c can mean: a, b, c, a and b, a and c, b and c, or a, b, and c, where each of a, b, and c can be an element or a collection containing one or more elements.

It should be noted that in the embodiments of this application, “equal to” can be used in conjunction with “greater than,” applicable to the technical solutions used when greater than, and can also be used with “less than,” applicable to the technical solutions used when less than. It should be clarified that when “equal to” is used with “greater than,” it is not used with “less than,” and vice versa. In the embodiments of this application, the terms “of,” “corresponding,” and “corresponding” can sometimes be used interchangeably, and it should be noted that when their differences are not emphasized, they convey the same meaning.

Firstly, an explanation of certain terms involved in the implementation examples of this application is provided for the understanding of those skilled in the art.

1. Digital Polymerase Chain Reaction (dPCR), is an absolute quantification technique for nucleic acid molecules. There are currently three methods for quantifying nucleic acid molecules: photometry, which is based on the absorbance of nucleic acid molecules; Real Time PCR, which is based on the Ct value, where the Ct value refers to the cycle number corresponding to the detectable fluorescence value; dPCR is the latest quantification technique, based on the single-molecule PCR method for counting nucleic acid quantification, and is an absolute quantification method. It mainly adopts the microfluidic or droplet-based methods, which are hot research areas in current analytical chemistry, dispersing the highly diluted nucleic acid solution into the microreactors or droplets on the chip, with the number of nucleic acid templates in each reactor being less than or equal to one. After the PCR cycles, reactors with a nucleic acid molecule template will emit a fluorescence signal, while those without a template will not. Based on the relative proportion and the volume of the reactors, the nucleic acid concentration of the original solution can be calculated.

2. Digital ELISA (Digital Enzyme Linked Immuno Sorbent Assay) is an absolute quantification technique for protein molecules. It enables ultra-high sensitivity detection of proteins directly in body fluids such as serum and plasma, promoting early disease detection, condition monitoring, and assisting in precision medication, thereby improving the quality of life and extending lifespan. The single-molecule immunoassay array technology is based on the ability to separate individual magnetic beads with immune complexes, using standard ELISA reagents. The main difference between Simoa technology and traditional immunoassays is that Simoa can capture individual molecules in femtoliter-sized wells, allowing for the digital readout of individual magnetic bead signals. This technology has an average sensitivity that is 1000 times higher than traditional ELISA, with a coefficient of variation (CVs) of less than 10%.

3. Real-time Quantitative Polymerase Chain Reaction (qPCR) has at least the following characteristics: it requires fewer instruments, using only one machine. The detection time is short, only 45 minutes to 1 hour and 10 minutes (depending on the reagent), while qPCR requires 3-4 hours, enzyme immunoassay endpoint quantification requires 6-8 hours, and fluorescence endpoint quantification requires 2-3 hours. The operation of fully automatic qPCR is extremely simple: after pretreatment, insert the sample into the instrument and a report can be generated on the computer after one hour, without the need to open the lid or move the sample (as with previous methods), avoiding contamination. The results are precise: qPCR can only provide qualitative results, which are very rough; endpoint qPCR is not accurate enough due to fluorescence detection only after 40 thermal cycles, leading to saturation of the measured fluorescence and a semi-quantitative state. In contrast, Real-time qPCR continuously detects changes in the fluorescence value of each sample at every moment of amplification, with a detection precision of 0.1 RLU and a discrimination rate of 99.7% between samples with 5,000 and 10,000 template copies.

Currently, dPCR (Digital Polymerase Chain Reaction) is a representative technology for single-molecule nucleic acid diagnostics, with the advantages of high sensitivity and absolute quantification. The existing technology mainly adopts microfluidic or droplet-based methods, which are hot research areas in current analytical chemistry, dispersing the highly diluted nucleic acid solution into the microreactors or droplets on the chip, with the number of nucleic acid templates in each reactor being less than or equal to one. After the PCR cycles, reactors with a nucleic acid molecule template will emit a fluorescence signal, while those without a template will not. Based on the relative proportion and the volume of the reactors, the nucleic acid concentration of the original solution can be calculated. In this article, the digital PCR method also includes the aforementioned droplet dispersion method, but uses isothermal amplification instead of temperature cycling amplification methods such as digital LAMP and digital RPA. However, the integration level of existing dPCR products is not high, requiring readers, scanners, precision optical components, or complex microfluidics and multiple machines to participate in the nucleic acid diagnostic process, making the instruments complex and performance unstable. DELISA (Digital Enzyme Linked Immuno Sorbent Assay) is a representative technology for single-molecule protein diagnostics. The typical feature of dELISA is that it allows testing in scenarios of low abundance or inconveniently obtained rare samples. Considering the current market products, typical technologies for immune assays include: traditional ELISA technology, radioimmunoassay technology, immunoturbidimetry, enzyme-enhanced chemiluminescence technology, electrochemiluminescence technology, etc. The typical characteristic of these technologies is that, generally, with a sample volume of 50 μL, the highest detection sensitivity for the target biomarker is 0.01 pg/mL. In contrast, dELISA technology can achieve a sensitivity in the fg/mL range, and combined with a series of research and exploration, it is expected to expand the number of clinical biomarkers from around 200 to over 2000, and the number of biomarkers for scientific research from around 2000 to over 10,000. Single-cell detection technology is a representative technology for single-factor cell diagnostics. It can accurately provide information on intracellular substances and biochemical reactions within cells, reflecting the specific relationships between cellular functions and chemical components. Since cells are very small and the content of intracellular components is generally in the fmol-zmol range, detectors applied in single-cell analysis should at least be able to monitor at the fmol level. Single-molecule cell detection can reach the detection limit of zmol, allowing for more specific identification of minute differences between each cell, providing a tool basis for further opening up new biomarker detection ranges for major diseases!

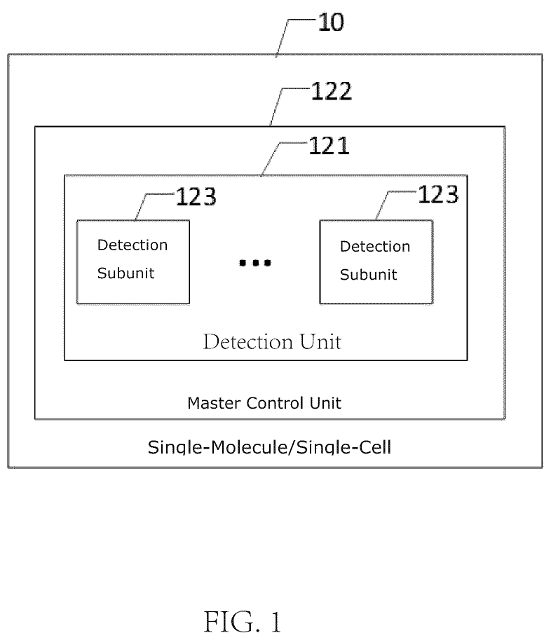

In response to the aforementioned issues, please refer to FIGS. 1, 2, 7, 8, 9, 10, and 11. This application provides a single-molecule/single-cell detection chip 10, which includes:

Microarray of Micropores, set on the surface of the single-molecule/single-cell detection chip, comprising multiple micropores 112. These micropores 112 are used to divide the test solution into multiple test droplets 15. The test droplets 15 include a reaction solution and at most one target nucleic acid molecule/protein molecule/cell 16. The target molecule 16 emits light when combined with the reaction solution.

Detection IC Circuit 11, located beneath the microarray of micropores, includes:

Detection Unit 121, comprising multiple detection subunits 123, each corresponding to one of the multiple micropores 112, connected to the main control unit 122. The detection subunits 123 are used to identify the target molecules in the test droplets 15. Test droplets 15 with a light intensity greater than the first threshold yield raw measurement results, which are sent to the main control unit 122.

Main Control Unit 122, responsible for power management, clock management, controlling the detection subunits 123, receiving the raw measurement results, generating the final detection results based on all the raw measurement results, and outputting the final detection results to the external circuit of the chip.

As an example, the microarray of micropores is made of insulating, inert materials, electrically isolating the test droplets 15 from the detection IC circuit 11. Specifically, the microarray of micropores is composed of negative photoresist, silicone, and other insulating, inert materials. It can be created on a CMOS wafer through methods such as single-crystal silicon etching, polycrystalline silicon deposition, polymer material coating, and pattern transfer and micromachining methods like photolithography, nanoimprinting, screen printing, dry etching, and laser etching to form the said micropores 112.

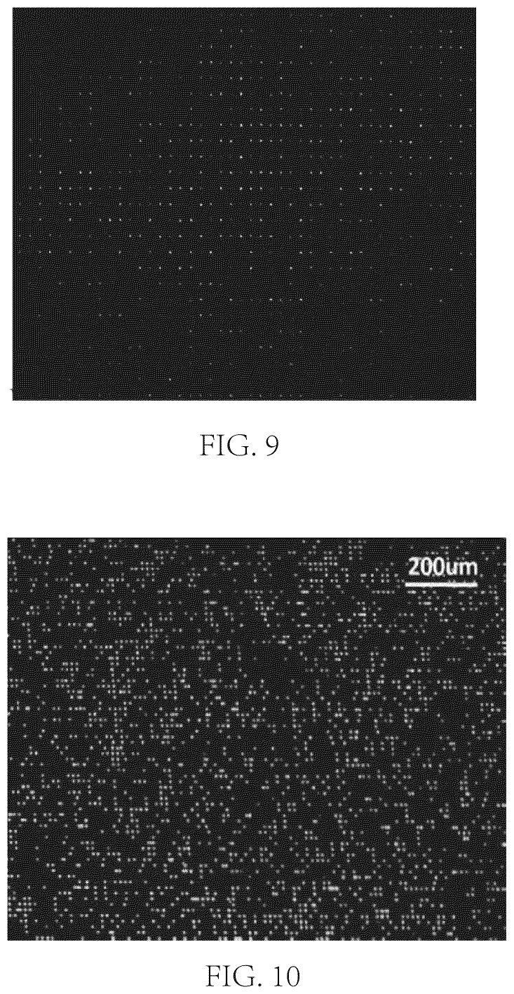

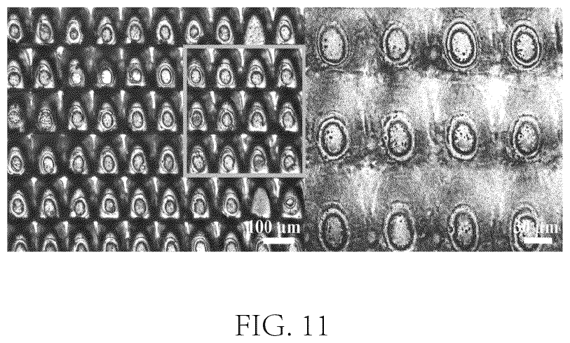

For example, the detection unit 121 can be one or multiple, and different biosensors can be set between multiple detection units 121 to achieve detection of different targets. These different targets include but are not limited to nucleic acid (DNA or RNA) molecules (FIG. 9), protein molecules (FIG. 10), cells (FIG. 11), peptides, or metabolites, etc.

For example, the target nucleic acid molecule is a DNA molecule or an RNA molecule.

For example, the raw measurement result is an analog signal, which indicates the presence of the target nucleic acid molecule/protein molecule/cell in the test droplet.

For example, in addition to the target molecule, the test droplet may also include non-target molecules, such as test reagent molecules, premixed liquids, etc. These non-target molecules can be nucleic acid molecules (FIG. 9)/protein molecules (FIG. 10)/cells (FIG. 11) or inorganic molecules, which vary with the specific reagent materials and are not uniquely defined here.

For example, the reaction solution may include nucleic acid molecules, protein molecules, cells, and inorganic molecules.

In specific implementations, the number of detection subunits 123 is set according to the number of micropores 112 in the microarray. One micropore 112 and one detection subunit 123 constitute a detection pixel point, achieving the detection of a target nucleic acid molecule/protein molecule/cell. The test solution is dropped onto the microarray of micropores, and the test solution is divided into multiple test droplets 15 through the multiple micropores 112. The test droplets 15 include a reaction solution and at most one target molecule 16. Then, the detection subunit 123 rapidly heats the test droplets 15, causing the target molecule 16 in the test droplets 15 with the target molecule 16 to rapidly amplify into the same multiple target molecules within the test droplets 15. Due to point heating, the heat does not disperse, which can reduce the reaction time of conventional PCR from 1-2 hours to a few minutes. The multiple target molecules obtained after amplification have sufficient fluorescence intensity, enabling the detection unit 121 to accurately identify the target test droplets 15 with the target molecule and generate and send the raw measurement results to the main control unit 122. The main control unit 122 summarizes all the raw measurement results and converts them into digital signals, which are then output to the external circuit of the chip.

It is evident that in this application, the single-molecule/single-cell detection chip 10 integrates functions such as amplification, recognition, and data processing into a single chip, simplifying the single-molecule instrument system, enhancing the reaction speed of instruments and reagents, and increasing stability. Based on CMOS-MEMS chip technology, it integrates five core functions-liquid sampling, droplet dispersion, nucleic acid/protein/cell reaction cycling, optical detection, and data processing-onto a silicon-based chip. This is compatible with low-cost, mature CMOS fabrication processes, significantly reducing the complexity of instruments and the cost of conventional microfluidic control, and features minimal usage, ultra-fast response, and high-sensitivity optical detection, achieving ultra-fast, fully automatic, high-throughput, and absolute quantification.

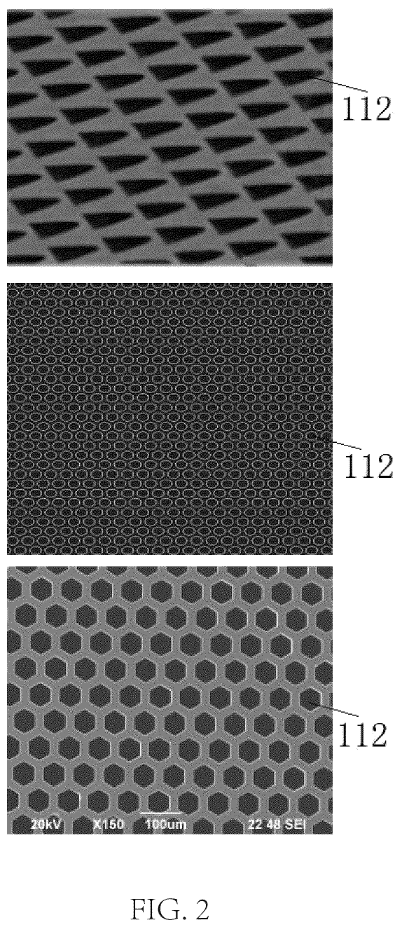

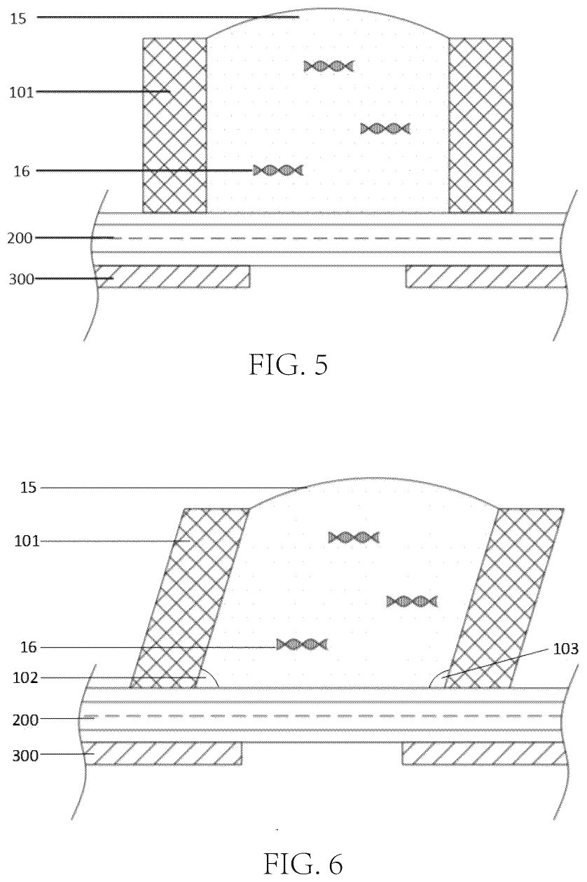

In some embodiments, please continue to refer to FIGS. 2, 5, 6, and 8, where multiple micropore arrays are orderly arranged on the surface of the single-molecule/single-cell detection chip to form a micropore array; all walls of the micropores 112 are perpendicular to the bottom of the micropores 112; or, all walls of the micropores 112 form an acute angle 102 or an obtuse angle 103 with the bottom of the micropores 112.

For example, the multiple micropore arrays can be orderly arranged in a square array, pyramid array, etc., without unique restriction.

For example, the micropores 112 are wedge-shaped, similar to the rim of a pitcher plant, which is conducive to the dispersion of droplets. It is understood that the micropores 112 can also be other shapes (such as circular, triangular, square, hexagonal, etc.), without unique restriction.

For example, the acute angle can be 30-90 degrees, and the obtuse angle can be 90-150 degrees.

It can be seen that in this embodiment, by designing the structure and arrangement of the micropores, the multiple micropores 112 more easily divide the test solution into test droplets.

In certain embodiments, the inner surface of the micropores 112 is hydrophilic, while the bottom of the micropores 112 can be either hydrophilic or hydrophobic. The top of the pore walls 101 is hydrophobic, facilitating the formation of the test droplets within the micropores 112. The micropores 112 can be etched from silicon wafers or can be made from curable polymer materials, formed through specific photolithography, etching, nanoimprinting, and other pattern transfer methods.

It is apparent that the inclined walls 101 of the micropores 112 allow the test solution to be more smoothly channeled into the micropores 112 on the microarray, subsequently forming the test droplets 15.

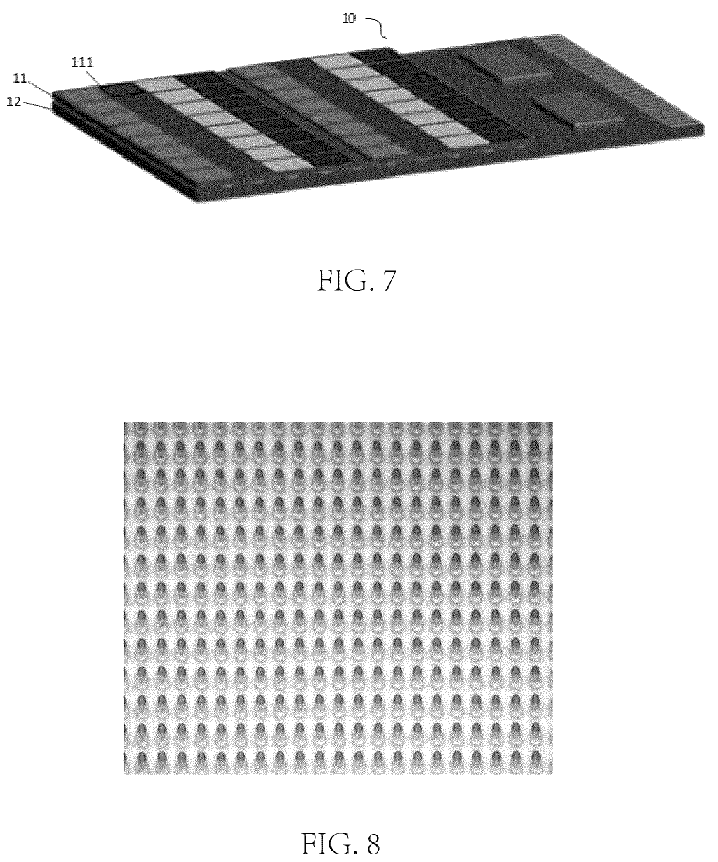

In some embodiments, as further detailed in FIG. 7, the microarray includes multiple droplet regions 111, with the multiple micropores 112 distributed over these droplet regions 111.

For example, each droplet region 111 can support a customized filter layer 1211 to achieve 1-6 color light detection. By setting different filter layers 1211 for each droplet region 111, a variety of light detections can be realized. It is understood that the types of light tests can be increased by adding more droplet regions 111.

It can be seen that in this embodiment, different light tests are simultaneously implemented on the same detection chip 10, thereby increasing the variety of simultaneous nucleic acid/protein/cell tests.

Of course, in dPCR, dELISA (single-molecule immunoassay), and single-cell analysis, the depths of the micropores are different. The depth of the micropores for dPCR typically ranges from 100-300 μm, for dELISA it is 3-5 μm often using 3 μm magnetic beads with micropores of 3-5 μm and for single-cell analysis, the micropore depth is usually 20-30 μm.

In some embodiments, the micropore array is made of inert materials and is rendered hydrophilic or hydrophobic through physical or chemical modification.

For example, the micropore array uses dense polymer materials or inert materials such as silicon and glass, which have low auto-fluorescence characteristics, high tolerance, and stability for the test solution. At temperatures ranging from 20° C. to 100° C., these materials do not affect the amplification reaction of the test solution in micropore 112.

It can be seen that in this embodiment, the micropore array is made of inert materials to withstand the erosion of the amplification reaction of the test solution.

In some embodiments, the bottom of micropore 112 is a transparent layer, allowing the light emitted by the test droplet 15 to pass through the bottom of micropore 112.

For example, the material for the transparent layer can be silicone, epoxy resin, or other transparent materials, without unique restriction.

In specific implementations, after opening multiple micropores 112 on the surface of the single-molecule/single-cell detection chip, through-holes are also opened at the bottom of the multiple micropores 112. These through-holes are then filled with transparent material to form a transparent layer.

It can be seen that in this embodiment, by setting a transparent layer at the bottom of micropore 112, the light stimulated by the target molecules can pass through the transparent layer and illuminate the detection subunit 123.

### Embodiment 1

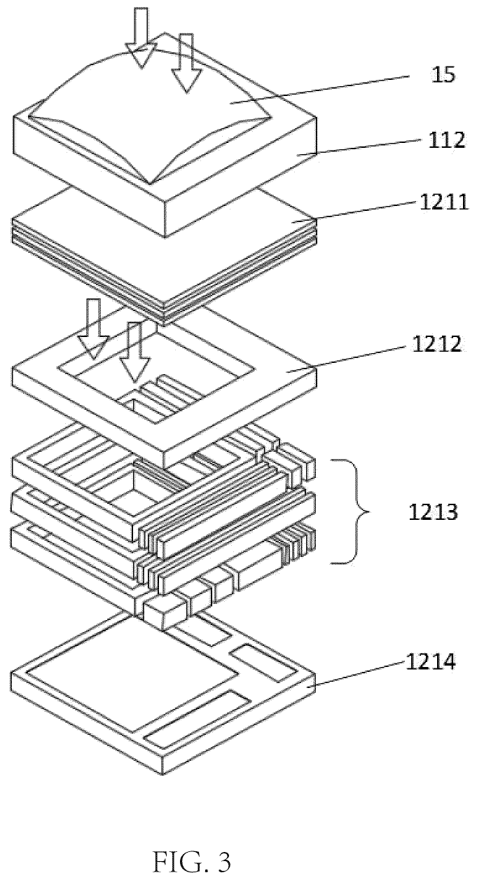

Refer to FIG. 3, the detection subunit 123 includes a stacked arrangement of filter layers 1211, heating electrodes, detection circuits 1214, and auxiliary circuits;

The filter layer 1211 is set below the corresponding micropore 112, composed of several sets of first and second refractive layers stacked together. It is used to filter the incident excitation light of the micropore 112 and allows the light emitted after droplet amplification, with a wavelength greater than that of the first and second refractive layers, to pass through the filter layer 1211 to reach the detection unit 121. The refractive index of the first refractive layer is different from that of the second refractive layer;

The heating electrode is set between the filter layer 1211 and the detection circuit 1214;

The detection circuit 1214 includes one or more photodetectors, which receive row and column gating instructions and control commands. When the photodetectors receive a light signal, they generate and send the raw measurement results to the main control unit 122;

The auxiliary circuit includes a temperature sensing circuit. The thermosensitive element of the temperature sensing circuit is set close to micropore 112 or within the main control unit 122. It is used to read the temperature signals of one or more detection circuits 1214 and outputs them through the main control circuit to the external circuit.

Preferably, the micropore 112 can be aligned and bonded to the CMOS chip using non-MEMS methods such as double-sided tape.

### Embodiment 2

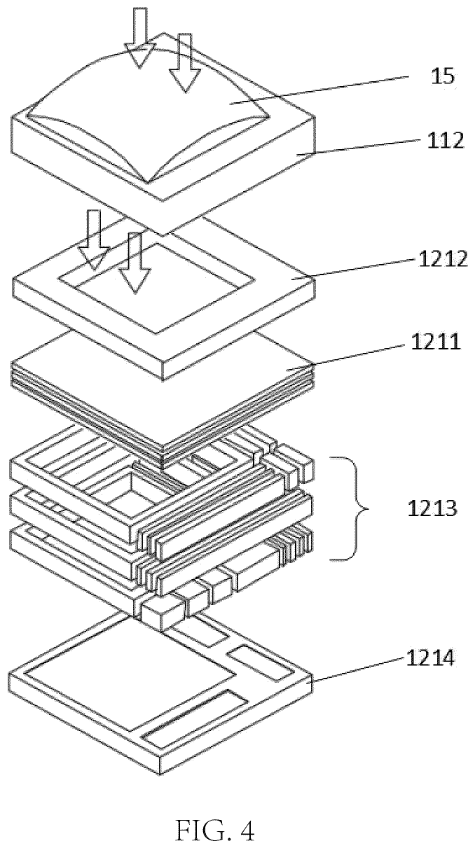

Please refer to FIG. 4, the heating electrode is set between the micropore 112 and the filter layer 1211. It is used to heat the test droplet to the target temperature for isothermal amplification, or to perform multiple temperature cycles for the variable temperature amplification reaction of nucleic acid/protein/cell molecules.

Preferably, the photodetector is a photodiode or avalanche diode.

Preferably, the first and second refractive layers are made of corresponding refractive materials, with the number of layers in the first refractive layer being no less than two, and the number of layers in the second refractive layer being no less than two.

Preferably, the photodetector uses a front-illuminated or back-illuminated circuit structure (i.e., front-illuminated CMOS or back-illuminated CMOS), thereby converting the light signal into an analog electrical signal.

Preferably, the filter layer 1211 is a filter or made of filtering material. The filter layers 1211 between detection subunits 123 can be the same or different and can be set according to the target molecules to be detected, without unique restriction.

Preferably, the heating electrode 1212 is a microelectrode, and the temperature is controlled by the main control unit 122 controlling the heating electrode 1212. Due to point-to-point temperature control, ultra-fast heating or cooling or isothermal amplification of the test droplet 15 is achieved, with a specific amplification speed of about 5-10 minutes.

Preferably, the detection circuit 1214 is a CMOS circuit, made using CMOS-compatible processes on a silicon base.

Preferably, the micropore 112 can be aligned and bonded to the CMOS chip using non-MEMS methods such as double-sided tape.

Preferably, the variable temperature amplification reaction can be polymerase chain reaction (PCR), etc.

Preferably, the isothermal amplification reaction can be polymerase chain reaction (PCR), protein reaction, cell reaction, etc.

It can be seen that in this application, the detection unit 121 can achieve single-molecule detection functionality through different setup methods. The detection circuit 1214 heats the test droplet 15 at a single point to reduce heating power consumption, improve heating efficiency, speed up the light testing time, and achieve the identification of target molecules. At the same time, the detection circuit 1214 is made using CMOS-compatible processes, which are cost-effective and have high quality controllability.

Continuing with the reference to FIGS. 3 and 4, the auxiliary circuit also includes multiple metal connection lines 1213. These metal connection lines 1213 are respectively set between the heating electrode 1212, the temperature sensor, and the detection circuit 1214, connecting the heating electrode 1212, the temperature sensor, and the detection circuit 1214 electrically to the main control unit 122.

For example, the metal connection lines 1213 can be printed metal lines.

For example, there can be multiple or multilayered metal connection lines 1213, which can be selected according to specific needs, without unique restriction.

It can be seen that in this embodiment, the metal connection lines 1213 establish electrical connections between the heating electrode 1212, the detection circuit 1214, and the main control unit 122.

In specific implementations, microlenses can be set on the filter layer 1211 or the heating electrode 1212 to converge the light emitted from micropore 112.

For example, the microlens can be a convex lens, which can be set above the filter layer 1211, below the heating electrode 1212, or below micropore 112, as long as the light signal first passes through the microlens to converge, then illuminates the filter layer 1211, without unique restriction.

It can be seen that in this embodiment, the microlens achieves the convergence of the light signal, making the light easier to be recognized.

In some embodiments, please refer to FIG. 3 or 4, the heating electrode 1212 has a first light-transmitting hole through which the light signal passes.

For example, the first light-transmitting hole can be square, circular, triangular, hexagonal, etc., without unique restriction.

For example, the electrode body of the heating electrode 1212 surrounds the first light-transmitting hole in a semi-enclosed or fully enclosed form.

It can be seen that in this embodiment, the first light-transmitting hole allows the heating electrode 1212 to perform its heating function without blocking the light signal directed towards the detection unit 121.

Please refer to FIG. 3 or 4, the detection circuit 1214 includes a substrate and photodetectors set on the substrate.

For example, the substrate can be a silicon substrate.

In specific implementations, the photodetector is set on the substrate and receives the light signal emitted by the target molecule through the first light-transmitting hole, filter layer 1211, and transparent layer.

For example, the photodetector can be a photodiode, avalanche diode, etc.

It can be seen that in this embodiment, the integration of the photodetector on the substrate is achieved.

In some embodiments, the main control unit 122 includes a power management circuit, clock management circuit, row and column selection circuit, signal readout circuit, signal processing circuit, and I/O interface circuit;

The power management circuit is used to convert the external power supply of the chip into one or more DC levels inside the chip;

The clock management circuit is used to receive and process the clock signal provided by the external chip as the time base for the internal digital circuit of the chip;

The row and column selection circuit, connected to the power management circuit, is used to send row and column gating instructions to gate the corresponding row and column positions of the detection subunit 123;

The signal readout circuit, connected to the power management circuit, is used to read all light signals passing through the filter layer 1211;

The signal readout circuit also includes a preprocessing circuit, which is connected to the main control unit 122, used to average and denoise the digital electrical signal multiple times, or to compress the signal;

The I/O interface circuit, connected to the signal readout circuit and the temperature sensing circuit, is used to input the external power supply, clock, control signals, etc., into the chip, and to transmit the digital signals from the signal readout circuit and the temperature signals from the temperature sensing circuit to the external circuit of the chip in the form of digital signals.

In the given examples, the power management circuit is connected to an external power source, converting the external supply into a DC level between 1.2V-5V for internal chip power. This ensures stable and consistent circuit working voltage and current during power-up, power-down, voltage fluctuations, and electromagnetic interference.

The signal readout circuit includes an ADC that converts the raw measurement results into digital signals.

The I/O interface circuit includes various interfaces and data lines, which can be printed metal lines or other types of connectors.

The micropores 112 are processed using MEMS technology compatible with CMOS processes.

In practice, the single-molecule/single-cell detection chip 10 can output the detection results to devices such as monitors or computers for display and/or processing through the I/O interface circuit.

The power management circuit controls all the supply voltages (i.e., DC levels) inside the single-molecule/single-cell detection chip. Finally, the final detection results are output to the external circuit of the chip through the I/O interface circuit, where the external circuit performs the corresponding data processing.

The row and column selection circuit gates the detection circuit within each detection subunit 123, enabling the photodetectors in the detection circuit to begin testing the test droplets.

It is evident from this implementation that various sub-circuits within the main control unit 122 control the overall detection process.

This application also provides a single-molecule/single-cell diagnostic system, which includes:

The detection chip 10 as described above;

A sample droplet adding device, used to add test droplets 15 on the micropore array of the single-molecule/single-cell detection chip 10.

For example, the sample droplet adding device includes a dropper and a first moving module, where the clamping tool on the first moving module holds the dropper to add the test solution on the micropore array.

It can be seen that in this embodiment, the detection IC circuit 11 integrates functions such as amplification, recognition, and data processing, simplifying the structure of the biomimetic detection chip 10, enhancing stability, and simultaneously achieving the addition of the test solution droplets.

In summary, the single-molecule/single-cell detection chip provided by this application includes: a micropore array, set on the surface of the single-molecule/single-cell detection chip, comprising multiple micropores 112, which are used to divide the test solution into test droplets that include only a single target molecule; detection IC circuit 11, set below the micropore 112 array, includes: detection unit 121, comprising multiple detection subunits 123, each corresponding to one of the multiple micropores 112, connected to the main control unit 122; the detection subunits 123 are used to amplify the target molecule in the test droplets, measure the light intensity of the target test droplets with the target molecule after amplification, and send the raw measurement results to the main control unit 122; the main control unit 122 is used for power management, clock management, controlling the detection subunits 123, receiving the raw measurement results, generating the final detection results based on all the raw measurement results, and outputting the final detection results to the external circuit of the chip. This application integrates functions such as amplification, recognition, and data processing through the detection chip, simplifying the structure of the biomimetic detection chip, enhancing stability, using mature CIS and MEMS processes, with low mass production costs and high quality controllability; integrating liquid sampling, droplet generation, photoelectric detection, and temperature control modules on a silicon base; easy to expand, capable of achieving high-throughput detection sample volume and number of optical channels by adding droplet regions.

It should be noted that the above examples are only used to illustrate the technical solutions of this application and are not intended to limit them. Although the application has been described in detail with reference to the foregoing examples, those of ordinary skill in the art should understand that they can still modify the technical solutions recorded in the foregoing examples or equivalently replace some of the technical features; and these modifications or replacements do not depart from the spirit and scope of the technical solutions of each embodiment of the application.

What is claimed is:

1. A single molecule/single cell detection chip, comprising:
Micropore array, set on the surface of the said single molecule/single cell detection chip, including multiple micropores. The multiple micropores are used to divide the test solution into multiple test target droplets. The test droplets include a reaction solution and at most one target nucleic acid molecule/protein molecule/cell. The target nucleic acid molecule/protein molecule/cell can emit fluorescence of a specific wavelength after certain biochemical reactions and modifications, which can be detected by a photoelectric detector.
Detection IC circuit, set below the said micropore array, it includes:
Detection unit, comprising multiple detection sub-units set one-to-one corresponding to the multiple micropores. The multiple detection sub-units are connected to the main control unit. The detection sub-units are used to identify test droplets in which the emitted light intensity of the target nucleic acid molecule/protein molecule/cell exceeds a first threshold, obtain raw measurement results, and send the raw measurement results to the main control unit.
Main control unit, used for power management, clock management, controlling the detection sub-units, receiving the raw measurement results, generating final detection results based on the raw measurement results, and outputting the final detection results to the external circuit of the chip.

2. The single molecule/single cell detection chip according to claim 1, wherein the multiple micropore arrays are orderly arranged on the surface of the said single molecule/single cell detection chip to form a micropore array; all the pore walls of the said micropores are perpendicular to the bottom of the said micropores; or all the pore walls of the said micropores form an acute or obtuse angle with the bottom of the said micropores.

3. The single molecule/single cell detection chip according to claim 1, wherein the micropore array includes multiple droplet areas, and the multiple micropores are distributed over the multiple droplet areas; the test solution flows along a predetermined direction and covers the multiple droplet areas to form a test droplet array.

4. The single molecule/single cell detection chip according to claim 1, wherein the micropore array is composed of materials such as negative photoresist, silicon dioxide, silicone, etc., which are insulating, inert, and compatible with specific biochemical reactions. It can be produced on a CMOS wafer through methods such as single-crystal silicon etching, wet etching, polysilicon deposition, polymer material coating, and pattern transfer and microfabrication methods such as lithography, nanoimprinting, screen printing, dry etching, and laser etching.

5. The single molecule/single cell detection chip according to claim 1, wherein the inner side surface of the micropores is hydrophilic, and the bottom of the micropores is hydrophilic or hydrophobic.

6. The single molecule/single cell detection chip according to claim 1, wherein the micropore array is made of inert materials and obtains hydrophilicity or hydrophobicity through physical modification or chemical modification.

7. The single molecule/single cell detection chip according to claim 1, wherein the sealing method within the said chip includes thermal bonding, silicon-silicon bonding, glue sealing, and intermediate layer material sealing, etc.

8. The single molecule/single cell detection chip according to claim 1, wherein the sealing methods are divided into physical sealing and chemical sealing, etc.;
Physical sealing includes oil encapsulating water, film sticking, tape, and thin-film encapsulation sealing, etc.;
Chemical sealing includes the transition from liquid phase to solid phase by paraffin, polymer etching sealing, chemical deposition sealing, etc.

9. The single molecule/single cell detection chip according to claim 1, wherein the detection sub-unit includes a stacked arrangement of a filter layer, heating electrode, detection circuit, and auxiliary circuit;
The filter layer is set below the corresponding micropore, composed of several sets of first and second refractive layers stacked together, used to filter the incident excitation light of the micropore. After the droplet amplification, the fluorescence emission light with a wavelength greater than the cutoff wavelength of the filter layer mostly passes through the filter layer to reach the detection unit, while the incident excitation light with a wavelength lower than the cutoff wavelength of the filter layer is mostly filtered out. The refractive index of the first refractive layer is different from that of the second refractive layer.
The heating electrode is set between the filter layer and the detection circuit or between the micropore and the filter layer, used to heat the test droplet to the target temperature for isothermal amplification, or to perform multiple temperature cycles for the variable temperature amplification reaction of nucleic acids.
The detection circuit includes one or more photodetectors, used to receive row and column gating instructions and control commands, so that the one or more photodetectors generate and send the raw measurement results to the main control unit when they receive the light signal.
The auxiliary circuit includes a temperature sensing circuit, with the thermosensitive element of the temperature sensing circuit set close to the micropore or inside the main control unit, used to read the temperature signals of one or more detection circuits and output them through the main control circuit to the external circuit.

10. The single molecule/single cell detection chip according to claim 6, wherein the auxiliary circuit also includes multiple metal connection lines, which are respectively set between the heating electrode, temperature sensor, and the detection circuit, respectively making the heating electrode, temperature sensor, and the detection circuit electrically connected to the main control unit.

11. The single molecule/single cell detection chip according to claim 1, wherein the filter layer or heating electrode is equipped with microlenses for converging the light emitted from the micropores.

12. The single molecule/single cell detection chip according to claim 1, wherein the main control unit includes a power management circuit, clock management circuit, row and column selection circuit, signal readout circuit, signal processing circuit, and I/O interface circuit;
The power management circuit is used to convert the external power supply of the chip into one or more DC levels inside the chip.
The clock management circuit is used to receive and process the clock signal provided externally to the chip as the time base for the internal digital circuit of the chip.
The row and column selection circuit is connected to the power management circuit and is used to send row and column gating instructions to select the corresponding row and column position of the detection sub-unit.
The signal readout circuit is connected to the power management circuit and is used to read the raw measurement results outputted by the detection circuit and convert the raw measurement results into digital electrical signals.
The signal readout circuit also includes a preprocessing circuit, which is connected to the main control unit and is used to perform multiple averaging and noise reduction on the digital electrical signals, or to compress the signals.
The I/O interface circuit is connected to the signal readout circuit and the temperature sensing circuit, and is used to input the external power supply, clock, control signals, etc., into the chip, and to transmit the digital electrical signals from the signal readout circuit and the temperature signals from the temperature sensing circuit to the external circuit of the chip in the form of digital signals.

13. The single molecule/single cell detection chip according to claim 12, wherein the micropores are processed based on MEMS technology compatible with CMOS processes, or using precisely machined regular micro-through-hole arrays, and are bonded and aligned to the main control unit, allowing the signal readout circuit to read the optical signals from the micropore array one by one.

14. The single molecule/single cell detection chip according to claim 1, wherein the detected light can be visible light, fluorescent luminophores, up conversion luminescence, rare earth element luminescence, or quantum dot luminescence.
